(19) United States
(12) Patent Application Publication
(10) Pub. No.: US 2005/0239200 A1
(43) Pub. Date: Oct. 27, 2005

(54) Devices for culturing anaerobic microorganisms and methods of using the same

(76) Inventors: Scott W. Beckwith, Greer, SC (US); Drew Ve Speer, Simpsonville, SC (US); E. Jeffery Rhodehamel, Greer, SC (US); Janet Rivett, Simpsonville, SC (US); Cynthia J. Knight, Easley, SC (US)

Correspondence Address: CRYOVAC, INC.;SEALED AIR CORP, P.O. BOX 464, DUNCAN, SC, 29334, US

(21) Appl. No.: 10/831,033

(22) Filed: Apr. 23, 2004

(51) Int. Cl. C12Q 1/04; C12M 1/14

(52) U.S. Cl. 435/299.1

(57) ABSTRACT

Culture devices for promoting the growth of microorganisms, especially anaerobic microorganisms, are disclosed. The culture devices include an oxygen scavenging material. In one aspect, the culture device includes a supporting substrate and a cover sheet affixed to at least one edge of the supporting substrate. At least one of the supporting substrate and cover sheet includes the oxygen scavenging material. In another aspect, the culture device is an article that includes a container, e.g. a pouch, including a film having an oxygen scavenging material, with a culture media placed within the container.

### FIELD OF THE INVENTION

The present invention generally relates to culture devices for growing microorganisms, and more particularly relates to culture devices including an oxygen scavenging material for growing anaerobic microorganisms, as well as methods of using the same.

### BACKGROUND OF THE INVENTION

Many bacteria are sensitive to oxygen and will not grow in its presence. It can be useful in various environments to determine the viability of such anaerobic microorganisms. For example, it can be important to determine if anaerobic microorganisms are present in food processing and/or packaging facilities. It can also be important to determine the presence of anaerobic microorganisms in medical environments, for example, to determine the presence of pathogens in diagnostic assays. As another example, water treatment facilities test water samples to determine the presence or absence of such microbes.

A variety of devices are available for culturing microorganisms. For example, microorganisms have long been cultured using Petri dishes. As known in the art, Petri dishes are round, shallow, flat bottomed dishes with a suitable medium for growth of the microorganism, such as agar and nutrients. The use of agar medium, however, can be inconvenient and time consuming. For example, agar medium must be sterilized, melted and cooled prior to addition of the sample.

In addition, it can be difficult to provide an environment suitable for culturing anaerobic microorganisms using Petri dishes. Because anaerobic microorganisms do not thrive in the presence of oxygen, cumbersome physical and chemical techniques can be required to grow such organisms. Typically, such devices must be modified, i.e., shaped or configured, to provide a physical barrier to the transmission of oxygen. See U.S. Pat. Nos. 6,429,008 and 6,204,051, both to Copeland et al., and U.S. Pat. No. 4,906,566 to Cullimore et al. These patents discuss attempts to limit or reduce the oxygen content of such devices, for example, by the use of specially configured lids and/or dishes. Such devices, however, can be expensive and typically are not disposable, thus limiting their use in various applications.

Other techniques have been developed that use chemical agents incorporated into an anaerobic culturing device to remove oxygen. Generally, such devices include a reducing agent incorporated into a gel or nutrient media. For example, U.S. Pat. No. 4,476,224 to Adler describes a nutrient media containing a hydrogen donor and sterile membrane fragments of bacteria having an electron transfer system to reduce oxygen to water. U.S. Pat. No. 2,348,448 to Brewer; U.S. Pat. No. 3,165,450 to Scheidt; U.S. Pat. No. 5,034,331 to Brewer; and U.S. Pat. No. 4,419,451 to Garner et al. describe Petri dishes with reducing agents in a culture medium to absorb oxygen. U.S. Pat. No. 3,338,794 to Bladel describes an anaerobic bacteria culturing device formed of oxygen impermeable film layers and a nutrient media between the films, which includes a reducing compound.

Other patents are directed to the use of a reducing agent placed in a separate compartment or pouch within a culturing device. See U.S. Pat. No. 4,904,597 to Inoue et al.; U.S. Pat. No. 4,605,617 to Kasugai; and U.S. Pat. No. 6,123,901 to Albert et al.; and JP 357086288.

These and other devices, however, can also be cost prohibitive and may not be readily disposable. These devices can also be cumbersome to assemble and/or use.

Petrifilm™ plates commercially available from 3M include a self-supporting substrate with a reconstitutable gelling and nutrient composition adhered thereto. Upon application of a liquid sample to a device, the gelling agent hydrates to form a gelatin medium useful for growing microorganisms contained in the liquid sample. Such devices can also include a transparent coversheet.

The coversheet can be selected to provide the necessary amount of oxygen transmission. For example, polyethylene films have relatively high oxygen permeability and are suitable for use as a coversheet in a Petrifilm™ device for culturing aerobic organisms. In contrast, polyester films have relatively low oxygen permeability, and thus are more suited for use in devices for culturing anaerobic microorganisms.

Even Petrifilm™ devices that include an oxygen impermeable coversheet are not typically suitable for growth of anaerobic bacteria. Typically the sample must be incubated inside an airtight chamber or pouch containing a substantially oxygen free atmosphere. This can be accomplished through evacuation and or gas flushing, or through the presence of an oxygen scavenging sachet. Such additional steps can be cumbersome and increase culture times and costs. Microorganisms are typically cultured at biological temperatures such as 30-40° C., especially 36° C. To determine anaerobic bacterial counts, it is important to remove oxygen from the culture medium rapidly at that temperature.

### DEFINITIONS

“Film” is used herein in its generic sense and can include a film, laminate, sheet, web, coating, or the like.

“Oxygen scavenger”, “oxygen scavenging”, and the like herein means a material, such as a composition, compound, film, film layer, coating, and the like which can consume, deplete or react with oxygen from a given environment. According to U.S. Pat. No. 5,350,622, oxygen scavengers are made of an ethylenically unsaturated hydrocarbon and transition metal catalyst. The ethylenically unsaturated hydrocarbon may be either substituted or unsubstituted. As defined herein, an unsubstituted ethylenically unsaturated hydrocarbon is any compound that possesses at least one aliphatic carbon-carbon double bond and comprises 100% by weight carbon and hydrogen. A substituted ethylenically unsaturated hydrocarbon is defined herein as an ethylenically unsaturated hydrocarbon which possesses at least one aliphatic carbon-carbon double bond and comprises about 50% -99% by weight carbon and hydrogen. A substituted or unsubstituted ethylenically unsaturated hydrocarbon can have two or more ethylenically unsaturated groups per molecule, such as three or more ethylenically unsaturated groups and a molecular weight equal to or greater than 1,000 weight average molecular weight.

Examples of unsubstituted ethylenically unsaturated hydrocarbons include, but are not limited to, diene polymers such as polyisoprene, (e.g., trans-polyisoprene) and copolymers thereof, cis and trans 1,4-polybutadiene, 1,2-polybutadienes, (which are defined as those polybutadienes possessing greater than or equal to 50% 1,2 microstructure), and copolymers thereof, such as styrene/butadiene copolymer and styrene/isoprene copolymer. Such hydrocarbons also include polymeric compounds such as polypentenamer, polyoctenamer, and other polymers prepared by cyclic olefin metathesis; diene oligomers such as squalene; and polymers or copolymers with unsaturation derived from dicyclopentadiene, norbornadiene, 5-ethylidene-2-norbornene, 5-vinyl-2-norbornene, 4-vinylcyclohexene, 1,7-octadiene, or other monomers containing more than one carbon-carbon double bond (conjugated or non-conjugated).

Examples of substituted ethylenically unsaturated hydrocarbons include, but are not limited to, those with oxygen-containing moieties, such as esters, carboxylic acids, aldehydes, ethers, ketones, alcohols, peroxides, and/or hydroperoxides. Specific examples of such hydrocarbons include, but are not limited to, condensation polymers such as polyesters derived from monomers containing carbon-carbon double bonds, and unsaturated fatty acids such as oleic, ricinoleic, dehydrated ricinoleic, and linoleic acids and derivatives thereof, e.g. esters. Such hydrocarbons also include polymers or copolymers derived from (meth)allyl (meth)acrylates. Suitable oxygen scavenging polymers can be made by trans-esterification. Such polymers are disclosed in U.S. Pat. No. 5,859,145 (Ching et al.) (Chevron Research and Technology Company), incorporated herein by reference as if set forth in full. The composition used may also comprise a mixture of two or more of the substituted or unsubstituted ethylenically unsaturated hydrocarbons described above. The hydrocarbon can have a weight average molecular weight of 1,000 or more, but an ethylenically unsaturated hydrocarbon having a lower molecular weight is usable, e.g. if it is blended with a film-forming polymer or blend of polymers.

Other oxygen scavengers which can be used in connection with this invention are disclosed in U.S. Pat. No. 5,958,254 (Rooney), incorporated by reference herein in its entirety. These oxygen scavengers include at least one reducible organic compound which is reduced under predetermined conditions, the reduced form of the compound being oxidizable by molecular oxygen, wherein the reduction and/or subsequent oxidation of the organic compound occurs independent of the presence of a transition metal catalyst. The reducible organic compound is preferably a quinone, a photoreducible dye, or a carbonyl compound that has absorbance in the UV spectrum.

An additional example of oxygen scavengers which can be used in connection with this invention are disclosed in PCT patent publication WO 99/48963 (Chevron Chemical et al.), incorporated herein by reference in its entirety. These oxygen scavengers include a polymer or oligomer having at least one cyclohexene group or functionality. These oxygen scavengers include a polymer having a polymeric backbone, cyclic olefinic pendent group, and linking group linking the olefinic pendent group to the polymeric backbone.

An oxygen scavenging composition suitable for use with the invention comprises:
(a) a polymer or lower molecular weight material containing substituted cyclohexene functionality according to the following diagram: 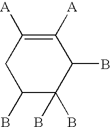
   where A may be hydrogen or methyl and either one or two of the B groups is a heteroatom-containing linkage which attaches the cyclohexene ring to the said material, and wherein the remaining B groups are hydrogen or methyl;
(b) a transition metal catalyst; and optionally
(c) a photoinitiator.

The compositions may be polymeric in nature or they may be lower molecular weight materials. In either case, they may be blended with further polymers or other additives. In the case of low molecular weight materials, they will beneficially be compounded with a carrier resin before use.

The oxygen scavenging composition of the present invention can include only the above-described polymers and a transition metal catalyst. However, photoinitiators can be added to further facilitate and control the initiation of oxygen scavenging properties. Suitable photoinitiators are known to those skilled in the art. Specific examples include, but are not limited to, benzophenone, and its derivatives, such as methoxybenzophenone, dimethoxybenzophenone, dimethylbenzophenone, diphenoxybenzophenone, allyloxybenzophenone, diallyloxybenzophenone, dodecyloxybenzophenone, dibenzosuberone, 4,4′-bis(4-isopropylphenoxy)benzophenone, 4-morpholinobenzophenone, 4-aminobenzophenone, tribenzoyl triphenylbenzene, tritoluoyl triphenylbenzene, 4,4′-bis(dimethylamino)benzophenone, acetophenone and its derivatives, such as, o-methoxy-acetophenone, 4′-methoxyacetophenone, valerophenone, hexanophenone, α-phenyl-butyrophenone, p-morpholinopropiophenone, benzoin and its derivatives, such as, benzoin methyl ether, benzoin butyl ether, benzoin tetrahydropyranyl ether, 4-o-morpholinodeoxybenzoin, substituted and unsubstituted anthraquinones, α-tetralone, acenaphthenequinone, 9-acetylphenanthrene, 2-acetyl-phenanthrene, 10-thioxanthenone, 3-acetyl-phenanthrene, 3-acetylindole, 9-fluorenone, 1-indanone, 1,3,5-triacetylbenzene, thioxanthen-9-one, isopropylthioxanthen-9-one, xanthene-9-one, 7-H-benz[de]anthracen-7-one, 1′-acetonaphthone, 2′-acetonaphthone, acetonaphthone, 2,4,6-trimethylbenzoyldiphenylphosphine oxide, bis(2,4,6-trimethylbenzoyl)phenylphosphine oxide, ethyl-2,4,6-trimethylbenzoylphenyl phosphinate, bis(2,6-dimethoxybenzoyl)-2,4,4-trimethylpentyl phosphine oxide, benz[a]anthracene-7,12-dione, 2,2-dimethoxy-2-phenylacetophenone, α,α-diethoxyacetophenone, α,α-dibutoxyacetophenone, 4-benzoyl-4′-methyl(diphenyl sulfide) and the like. Single oxygen-generating photosensitizers such as Rose Bengal, methylene blue, and tetraphenylporphine as well as polymeric initiators such as poly(ethylene carbon monoxide) and oligo[2-hydroxy-2-methyl-1-[4-(1-methylvinyl)phenyl]propanone] also can be used. The amount of photoinitiator can depend on the amount and type of cyclic unsaturation present in the polymer, the wavelength and intensity of radiation used, the nature and amount of antioxidants used, and the type of photoinitiator used.

Also suitable for use in the present invention is the oxygen scavenger of U.S. Pat. No. 6,255,248 (Bansleben et al.), incorporated herein by reference in its entirety, which discloses a copolymer of ethylene and a strained, cyclic alkylene, preferably cyclopentene; and a transition metal catalyst.

Another oxygen scavenger which can be used in connection with the invention is the oxygen scavenger of U.S. Pat. No. 6,214,254 (Gauthier et al.), incorporated herein by reference in its entirety, which discloses ethylene/vinyl aralkyl copolymer and a transition metal catalyst.

Thus, the oxygen scavenger can comprise at least one of:
i) oxidizable organic compound and a transition metal catalyst;
ii) ethylenically unsaturated hydrocarbon and a transition metal catalyst;
iii) a polymer having a polymeric backbone, cyclic olefinic pendent group, and linking group linking the olefinic pendent group to the polymeric backbone;
iv) a copolymer of ethylene and a strained, cyclic alkylene;
v) ethylene/vinyl aralkyl copolymer; and
vi) a photoreducible organic compound that has absorbance in the UV spectrum.

As indicated above, the oxygen scavenging polymer is combined with a transition metal catalyst. Suitable metal catalysts are those which can readily interconvert between at least two oxidation states.

The catalyst can be in the form of a transition metal salt, with the metal selected from the first, second or third transition series of the Periodic Table. Suitable metals include, but are not limited to, manganese II or III, iron II or III, cobalt II or III, nickel II or III, copper I or II, rhodium II, II or IV, and ruthenium II or III. The oxidation state of the metal when introduced is not necessarily that of the active form. Suitable counterions for the metal include, but are not limited to, chloride, acetate, stearate, palmitate, caprylate, linoleate, tallate, 2-ethylhexanoate, neodecanoate, oleate or naphthenate. Examples of useful salts include cobalt (II) 2-ethylhexanoate, cobalt stearate, and cobalt (II) neodecanoate. The metal salt may also be an ionomer, in which case a polymeric counterion is employed. Such ionomers are well known in the art.

Any of the above-mentioned oxygen scavengers and transition metal catalyst can be further combined with one or more polymeric diluents, such as thermoplastic polymers which are typically used to form film layers for use, for example, in plastic packaging articles. Well known thermosets can also be used as the polymeric diluent.

Further additives can also be included in the oxygen scavenging composition to impart properties desired for the device being manufactured. Such additives include, but are not limited to, fillers, pigments, dyestuffs, antioxidants, stabilizers, processing aids, plasticizers, fire retardants, anti-fog agents, etc.

The mixing of the components listed above can be accomplished by melt blending at a temperature in the range of 50° C. to 300° C. However, alternatives such as the use of a solvent followed by evaporation may also be employed. The blending may immediately precede the formation of the film layer(s) or precede the formation of a feedstock or masterbatch for later use in the production of finished culture devices. When the blended composition is used to make film layers, coextrusion, solvent casting, injection molding, stretch blow molding, orientation, thermoforming, extrusion coating, coating and curing, lamination or combinations thereof would typically follow the blending.

“Barrier,” “oxygen barrier”, and the phrase “barrier layer,” as applied to films and/or layers, is used herein with reference to the ability of a film, layer, or coating to serve as a barrier to one or more gases. High oxygen barrier multilayer films and laminates can be made from materials having an oxygen permeability, of the barrier material, less than 500 cm³ O₂/m²·day·atmosphere (tested at 1 mil thick and at 25° C. according to ASTM D3985), e.g. less than 100, less than 50, and less than 25 cm³ O₂/m²·day·atmosphere such as less than 10, less than 5, and less than 1 cm³ O₂/m²·day·atmosphere.

Oxygen (i.e., gaseous O₂) barrier layers can include, for example, ethylene/vinyl alcohol copolymer (EVOH), polyvinylidene chloride (PVDC), vinylidene chloride/methyl acrylate copolymer, polyvinyl chloride, polyalkylene carbonate, polyamide, polyethylene naphthalate, polyethylene terephthalate (PET), polyester, polyacrylonitrile, HDPE, polypropylene, ethylene/cyclic olefin copolymers, metal foils, SiOx compounds, oxide coated webs and mixtures thereof, and the like as known to those of skill in the art. In the present invention the O₂-barrier layer can beneficially include either EVOH or polyvinylidene chloride, the PVDC comprising a thermal stabilizer (i.e., HCl scavenger, e.g., epoxidized soybean oil) and a lubricating processing aid, which, for example, comprises one or more acrylates.

“EVOH” herein refers to the saponified product of ethylene/vinyl ester copolymer, generally of ethylene/vinyl acetate copolymer, wherein the ethylene content is typically between 20 and 60 mole % of the copolymer, and the degree of saponification is generally higher than 85%, preferably higher than 95%.

“Polyamide” as used herein refers to polymers having amide linkages along the molecular chain, such as synthetic polyamides such as nylons. Furthermore, such term encompasses both polymers comprising repeating units derived from monomers, such as caprolactam, which polymerize to form a polyamide, as well as polymers of diamines and diacids, and copolymers of two or more amide monomers, including nylon terpolymers, sometimes referred to in the art as “copolyamides”. “Polyamide” specifically includes those aliphatic polyamides or copolyamides commonly referred to as e.g. polyamide 6 (homopolymer based on ε-caprolactam), polyamide 6,6 (homopolycondensate based on hexamethylene diamine and adipic acid), polyamide 6,9 (homopolycondensate based on hexamethylene diamine and azelaic acid), polyamide 6,10 (homopolycondensate based on hexamethylene diamine and sebacic acid), polyamide 6,12 (homopolycondensate based on hexamethylene diamine and dodecandioic acid), polyamide 11 (homopolymer based on 11 -aminoundecanoic acid), polyamide 12 (homopolymer based on ω-aminododecanoic acid or on laurolactam), polyamide 6/12 (polyamide copolymer based on ε-caprolactam and laurolactam), polyamide 6/6,6 (polyamide copolymer based on ε-caprolactam and hexamethylenediamine and adipic acid), polyamide 6,6/6,10 (polyamide copolymers based on hexamethylenediamine, adipic acid and sebacic acid), modifications thereof and blends thereof. The term polyamide also includes crystalline or partially crystalline, or amorphous, aromatic or partially aromatic, polyamides. Examples of partially crystalline aromatic polyamides include meta-xylylene adipamide (MXD6), copolymers such as MXD6/MXDI, and the like. Examples of amorphous, semi-aromatic polyamides nonexclusively include poly(hexamethylene isophthalamide-co-terephthalamide) (PA-6,I/6T), poly(hexamethylene isophthalamide) (PA-6,I), and other polyamides abbreviated as PA-MXDI, PA-6/MXDT/I, PA-6,6/6I and the like.

Alternatively, metal foil, metal oxide, or SiOx compounds can be used to provide low oxygen transmission to a film and articles incorporating the same as a component. Metalized films can include a sputter coating or other application of a metal layer to a paperboard or polymeric substrate such as high density polyethylene (HDPE), ethylene/vinyl alcohol copolymer (EVOH), polypropylene (PP), polyethylene terephthalate (PET), polyethylene naphthenate (PEN), and polyamide (PA). The term “high density polyethylene” (HDPE) as used herein refers to a polyethylene having a density of between 0.94 and 0.965 grams per cubic centimeter.

Alternatively, oxide coated webs (e.g. aluminum oxide or silicon oxide) can be used to provide low oxygen transmission to a film used in connection with the invention. Oxide coated foils can include a coating or other application of the oxide, such as alumina or silica, to a polymeric substrate such as high density polyethylene (HDPE), ethylene/vinyl alcohol copolymer (EVOH), polypropylene (PP), polyethylene terephthalate (PET), polyethylene naphthenate (PEN), and polyamide (PA).

Even a sufficiently thick layer of a polyolefin such as HDPE, LLDPE, polypropylene, propylene copolymer, cyclic/olefin copolymer (COC), or PVC (polyvinyl chloride) can in some instances provide a sufficiently low oxygen transmission rate for the overall film to be effective as an oxygen barrier for this invention. The exact oxygen permeability optimally required for a given application can readily be determined through experimentation by one skilled in the art.

“Adhered” is inclusive of films which are directly adhered to one another via coextrusion, a heat-seal or other means, as well as films which are adhered to one another using an adhesive which is between the two films. As used herein, the phrase “directly adhered”, as applied to layers, is defined as adhesion of the subject layer to the object layer, without a tie layer, adhesive, or other layer therebetween. In contrast, as used herein, the word “between”, as applied to a layer expressed as being between two other specified layers, includes both direct adherence of the subject layer to the two other layers it is between, as well as a lack of direct adherence to either or both of the two other layers the subject layer is between. Thus, one or more additional layers can be imposed between the subject layer and one or more of the layers the subject layer is between.

“Tie” layer is used herein to refer to any internal layer having the primary purpose of adhering two layers to one another. In one embodiment, tie layer(s) can include any polymer having a polar group grafted thereon, so that the polymer is capable of covalent bonding to polar polymers such as polyamide and ethylene/vinyl alcohol copolymer. Exemplary polymers for use in tie layers include, but are not limited to, ethylene/unsaturated acid copolymer, ethylene/alkyl acrylate, ethylene/unsaturated ester copolymer, anhydride-grafted polyolefin, polyurethane, and mixtures thereof.

“Trigger” and the like herein means that process defined in U.S. Pat. No. 5,211,875, whereby oxygen scavenging is initiated (i.e. activated) by exposing an article such as a film to actinic radiation, having a wavelength of about 200 to about 750 nm at an intensity of at least about 1.6 mW/cm² or an electron beam at a dose of at least 0.2 megarads (MR), up to 20 megarads, wherein after initiation the oxygen scavenging rate of the article is at least about 0.05 cc oxygen per day per gram of oxidizable organic compound for at least two days after oxygen scavenging is initiated. Other sources of radiation include ionizing radiation such as gamma, x-rays and corona discharge. The duration of exposure can vary and generally depends on various factors such as but not limited to the amount and type of photoinitiator present, thickness of layers to be exposed, the wavelength and intensity of the radiation, and the like. An exemplary method provides a short “induction period” (the time that elapses, after exposing the oxygen scavenging component to a source of actinic radiation, before initiation of the oxygen scavenging activity begins) so that the oxygen scavenging component can be activated at or immediately prior to inoculation. Thus, “trigger” refers to exposing an article to actinic radiation as described above; “initiation” refers to the point in time at which oxygen scavenging actually begins or is activated; and “induction time” refers to the length of time, if any, between triggering and initiation. Reference is also made to U.S. Pat. No. 6,287,481, the entire disclosure of which is hereby incorporated by reference.

### BRIEF SUMMARY OF THE INVENTION

The present invention eliminates the cumbersome, time consuming and costly steps of culturing an anaerobic bacteria sample within an airtight chamber or pouch or in the presence of an oxygen scavenging sachet. The present invention provides an anaerobic culture device that includes as a component an activatable oxygen scavenging material. The culture device of the invention can be triggered to scavenge oxygen by exposing the device to actinic radiation.

In one embodiment of the invention, the culture device for growing anaerobic microorganisms includes a supporting substrate having opposing inner and outer surfaces and a cover sheet having opposing inner and outer surfaces affixed to at least one edge of the inner surface of the supporting substrate. The cover sheet is positioned to cover at least a portion, typically the majority of, and beneficially all of, the supporting substrate. At least one of the supporting substrate, the cover sheet, or both, includes an oxygen scavenger.

Any of the herein disclosed oxygen scavenging materials can be useful in the production of the culture devices of the invention.

The oxygen scavenger can be present, for example, as a film layer or coating in the supporting substrate, the cover sheet, or both the supporting substrate and the cover sheet. Alternatively the oxygen scavenger is present as a discrete monolayer or multilayer film. In one advantageous embodiment of the invention, the supporting substrate includes a multilayer film including at least one film layer comprising the oxygen scavenger, optionally adhered to an additional supporting substrate such as a polyethylene coated paper substrate, and the cover sheet comprises a transparent polymeric film.

The multilayer film can include a first outer oxygen scavenging film layer and a second outer film layer that includes an oxygen barrier. The oxygen scavenging layer and the oxygen barrier layer can be adhered directly to one another. Alternatively the oxygen scavenging layer and the oxygen barrier layer can be adhered to one another via one or more intermediate adhesive layers. Exemplary adhesives includes, for example, ethylene/unsaturated acid copolymers, ethylene/unsaturated ester copolymers, anhydride-grafted polyolefins, polyurethanes, and mixtures thereof.

The devices of the invention can further include medium suitable for supporting the growth of anaerobic microorganisms. For example, the device can include at least one gelling agent and at least one nutrient. Typically the medium for supporting the growth of anaerobic microorganisms is present along an inner surface of the supporting substrate. Alternatively the growth medium can be present along an inner surface of the cover sheet, or along an inner surface of both the supporting substrate and the cover sheet. The gelling agent and nutrient can be in the form of a reconstitutable powder and can be coated directly onto the device or optionally adhered to the device using a suitable adhesive.

In another embodiment of the present invention, the culture device for culturing anaerobic microorganisms comprises a container, such as a pouch or an end or side seal bag, that includes at least one film layer comprising an oxygen scavenger as a component thereof. In this aspect of the invention, a package of the invention includes the container, and further includes an anaerobic culture placed therein. The container is optionally sealed (and may be optionally gas flushed and/or vacuumized prior to sealing) to minimize flow of oxygen into the container.

The container can include a monolayer film layer comprising the oxygen scavenger. Alternatively the container can include a multilayer film that includes at least one oxygen scavenging layer and at least one oxygen barrier layer. In this aspect of the invention, the oxygen scavenging layer and the oxygen barrier layer can be directly adhered to one another or adhered via one or more adhesive layers disposed therebetween as discussed above.

In use, the container is exposed to actinic radiation at a dosage sufficient to trigger the oxygen scavenging material. A suitable culture device for growing anaerobic microorganisms is inoculated with a sample, and the inoculated device is placed within the container. The culture device inserted into the container can also optionally include an oxygen scavenger as a component thereof In this aspect of the invention, the oxygen scavenger of the culture device placed within the container can be triggered by exposure to actinic radiation, and the triggered device inoculated. Alternatively, the inoculated culture device placed within the container can be a device that does not inherently exhibit oxygen scavenging properties, such as a Petri dish, thin film culture device, and the like. Thus, the oxygen scavenger can be present in the container, the culture device, or both.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a top perspective view partially in section of an embodiment of a device useful for culturing or growing anaerobic microorganisms in accordance with the present invention;

FIG. 2 is a cross sectional view of a multilayer film useful as a component in the device for growing anaerobic microorganisms;

FIG. 3 is a top view of the device of FIG. 1 showing a grid pattern printed thereon;

FIG. 4 is a top perspective view of another embodiment of a device useful for culturing or growing anaerobic microorganisms in accordance with the present invention in which the device is in the form a pouch;

FIG. 5 is a top perspective view of another embodiment of a device useful for culturing or growing anaerobic microorganism in accordance with the present invention in which the device is in the form of an end seal bag;

FIG. 6 is a top perspective view of yet another embodiment of a device useful for culturing or growing anaerobic microorganisms in accordance with the present invention in which the device is in the form of a side seal bag;

FIG. 7 is a top perspective view partially in section of another embodiment of a device useful for culturing or growing anaerobic microorganisms in accordance with the present invention;

FIG. 8 is a top perspective view of the device of FIG. 7, shown in a closed condition;

FIG. 9 is a schematic view of process steps for culturing microorganisms, especially anaerobic microorganisms; and

FIG. 10 is a perspective view of an apparatus for triggering a device of the invention.

### DETAILED DESCRIPTION OF THE INVENTION

The present invention will now be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

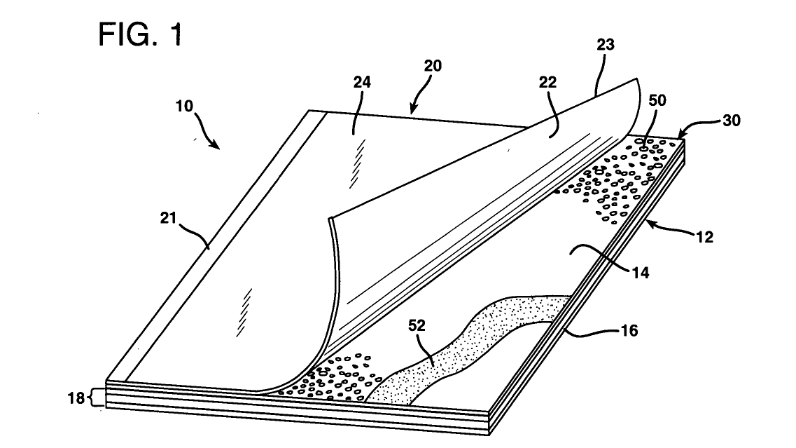

FIG. 1 is a top perspective view in partial section of an embodiment of the anaerobic culture device of the present invention. The anaerobic culture device, designated generally as 10, includes a supporting substrate 12 with an inner surface 14 opposing an outer surface 16. Culture device 10 also includes a cover sheet 20, which includes opposing inner surface 22 and outer surface 24. Cover sheet 20 is optionally fixed to one or more edges, such as edge 21, along the inner surface of supporting substrate 12. Cover sheet 20 is positioned so as to cover at least a portion, and typically substantially all, or entirely all, of the inner surface 14 of supporting substrate 12. As shown in FIG. 1, the edge 23 of cover sheet 20 is shown as peeled partially back to reveal the various components of supporting substrate 12.

At least one of supporting substrate 12, cover sheet 20, or both, include an oxygen scavenging material. Whether present in supporting substrate 12, or cover sheet 20, or both, the oxygen scavenging material can be in the form of a film layer or coating and can be continuous or discontinuous. The film can be used as a component in a rigid, semi-rigid, or flexible product, and can be adhered to another polymeric substrate and/or to a non-polymeric or non-thermoplastic substrate such as paper or metal.

The oxygen scavenging material can be in the form of a monolayer film (e.g. an extruded film or a coating). Alternatively, the oxygen scavenging material can be in the form of a multilayer film. The multilayer film can include at least two, and typically at least three, and up to twenty, or more, layers, at least one of which includes the oxygen scavenging material. One exemplary multilayer film is illustrated in FIG. 1 as three layer film 18 of supporting substrate 12.

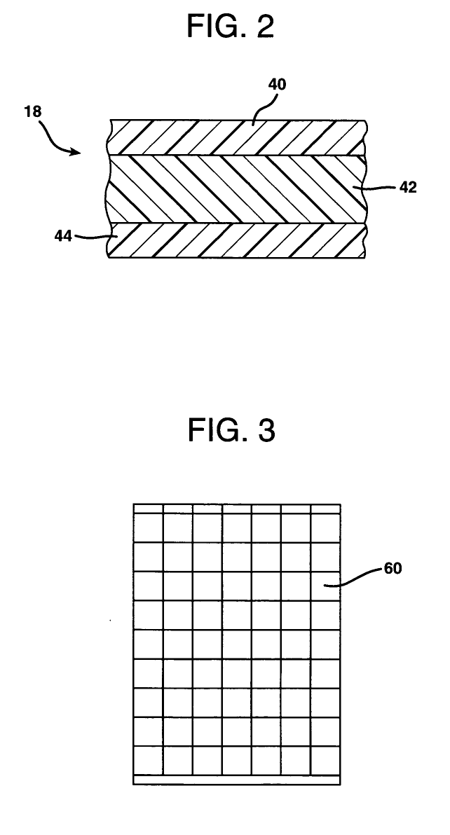

FIG. 2 is a cross sectional view of a three layer film 18 including an oxygen scavenger. As illustrated in FIG. 2, multilayer film 18 includes a first outer layer 40, an intermediate layer 42 and a second outer layer 44. Outer layer 40 is adjacent substrate 30 (of the device of FIG. 1) so that layer 40 is sandwiched between substrate 30 and inner layer 42.

One embodiment of the invention includes a first outer layer 40 including an oxygen scavenging material. A second layer 42 serves as an adhesive and layer 44 is effective as an oxygen barrier layer. Oxygen scavengers suitable for commercial use in monolayer and multilayer films and laminates of the present invention are disclosed herein, and e.g. in U.S. Pat. No. 5,350,622, and a method of initiating oxygen scavenging generally is disclosed in U.S. Pat. No. 5,211,875. Suitable equipment for initiating oxygen scavenging is disclosed in U.S. Pat. No. 6,287,481 (Luthra et al.). These patents are incorporated herein by reference in their entirety.

A second embodiment of the invention includes a first outer layer 40 of polyolefin, a second layer 42 that includes an oxygen scavenging material, and a third layer 44 that is effective as an oxygen barrier.

Second outer layer 44 is effective as an oxygen barrier layer.

In FIG. 2, first outer layer 40 and second outer layer 44 can be adhered to one another.

For example, as illustrated in FIG. 2, three layer film component 18 can include intermediate layer 42. When present, intermediate layer 42 can include an adhesive material to adhere oxygen scavenging layer 40 and oxygen barrier layer 44. Intermediate layer 42 can also be referred to as a tie layer.

Returning again to FIG. 1, device 10 can further include media suitable for culturing an anaerobic microorganism. For example, upper surface 14 of supporting substrate 12 can include a layer of culture medium 50, which can be dried to provide a dry medium on supporting substrate 12. Culture medium 50 can be in the form of a coating or a coating with discrete particulates. Alternatively, a layer of adhesive 52 may be coated on supporting substrate 12, which serves to hold a culture medium that may be applied as a powder.

FIG. 1 illustrates the culture medium and optional adhesive as components of supporting substrate 12 only. A suitable culture medium and optional adhesive, however, can alternatively be present on inner surface 22 of cover sheet 20. Alternatively both supporting substrate 12 and cover sheet 20 can include a suitable culture medium and optional adhesive on inner surfaces 14 and 22, respectively.

Culture medium 50 can include one or more dry gelling agent(s) and/or nutrient(s) suitable for supporting bacterial growth. Advantageously the dry gelling agent(s) and/or nutrient(s) are present as a substantially uniform monolayer across a substantial portion, generally across substantially all, of inner surface 14, or inner surface 24, or both, for easy hydration.

The majority of the components making up culture medium 50 are typically hydratable. That is, culture medium 50 can include powders of appropriate gelling agents and/or nutrients such that the addition of water to culture medium 50 reconstitutes the powders to create a suspension thereof.

Suitable gelling agents for inclusion in powder form include both natural and synthetic gelling agents that form solutions in water at room temperature or up to about 40° C., depending on the temperature of the liquid sample that is added to the device. Gelling agents such as hydroxyethyl cellulose, carboxymethyl cellulose, polyacrylamide, locust bean gum and algin form solutions in water at room temperature and are suitable gelling agents for providing water hydratable powders or solids, according to this invention. Other useful gelling agents in powder form are agar, guar gum and xanthan gum. The gelling agents are useful individually, or in combination with other gelling agent(s).

Suitable nutrients for supporting bacterial growth are known in the art and include without limitation yeast extract, peptone, sugars, suitable salts, and the like. An example is known as Standard Methods Nutrients described in Standard Methods for the Examination of Dairy Products, 14^{th} Edition, American Public Health Association, Washington D.C. Those skilled in the art will recognize that a variety of other formulations could be used and that these do not detract from the scope of this invention.

Culture medium can include gelling agent(s) only. When culture medium 50 includes only a gelling agent, the end user can incorporate nutrients suitable for growth of a particular bacteria in a bacterial sample to be cultured. If nutrient is incorporated with the gelling agent, the dry powdered nutrients can be incorporated directly in the powder or suspended in a rapidly-evaporating liquid such as ethanol, or the like. In other instances, dry powdered nutrients can be suspended or dissolved in aqueous solutions. An aliquot of the liquid can be added to inner surface 14 of supporting substrate 12 that has been previously coated with adhesive and gelling agent. The liquid is allowed to evaporate, under sterile condition, leaving ample nutrients along with the gelling agent.

The adhesive, when present, can be sufficiently transparent when hydrated to allow viewing of the colonies of microorganisms growing on the surface of the substrate through the coated substrate and/or the cover sheet. The adhesive can also be coated on the supporting substrate in a thickness that allows the substrate to be uniformly coated with dry culture medium without completely embedding the medium in the adhesive. Adhesive compositions that turn opaque upon exposure to water can also be used, for example, where colony visualization is not required.

When present, adhesive 52 can be water insoluble and does not inhibit the growth of the bacteria to be added to the device. Advantageously adhesive 52 is a pressure-sensitive adhesive, for example, a pressure-sensitive adhesive comprising a copolymer of an alkyl acrylate monomer and an alkyl amide monomer, with a weight ratio of alkyl acrylate monomer to alkyl amide monomer from about 90:10 to 99:1, more typically 95:5 to 98:2. Heat-activated adhesives having a lower melting substance coated onto a higher melting substance and/or water-activated adhesives such as mucilage are also known and can be used in this invention.

As noted above, adhesives are not required. For example, it is possible to dissolve or suspend a powder, for example of a dry gelling agent and/or nutrients, in a liquid. The liquid can be coated onto inner surface 14 of supporting substrate 12 and dried to provide a coating of dry powder on the surface.

Examples of various gelling, nutrient and/or adhesive compositions useful in the culture devices of the present invention are described, for example, in U.S. Pat. Nos. 5,232,838; 4,565,783; 5,869,321; 5,443,963; 5,462,860; 5,958,675; 5,089,413; and 5,601,998, and U.S. patent application Publication 2002/0110906 A1, all incorporated by reference herein in their entirety.

When using culture media device 10 illustrated in FIG. 1, an accurate count of the colonies of microorganisms present can be desirable. As illustrated in FIG. 3, the counting of colonies of microorganisms, such as bacteria colonies, can be facilitated by imprinting square grid pattern 60 on substrate 12 or cover sheet 22 by any suitable printing method.

To further aid in the visualization of bacterial colonies, it may be desirable to incorporate a dye into the medium mixture or alternatively into the adhesive. Suitable dyes are those which are metabolized by the growing microorganisms, and which cause the colonies to be colored for easier visualization. Examples of such dyes include triphenyl tetrazolium chloride, p-tolyl tetrazolium red, tetrazolium violet, veratryl tetrazolium blue and related compounds. Other dyes sensitive to pH changes such as neutral red are also suitable.

In use, a predetermined amount of inoculum, typically about 1 to 5 ml (e.g., 2-3 ml) of inoculum, is added to the device illustrated in FIG. 1 by pulling back cover sheet 20 and adding the inoculum (e.g., an aqueous microbial suspension) to the middle of culture medium 50. Cover sheet 20 is then replaced over substrate 12 and the inoculum is evenly spread on the substrate.

As the inoculum contacts and is spread on substrate 12, the culture medium on substrate 12 hydrates to form a growth-supporting nutrient gel. The inoculated device is then incubated for a predetermined time after which the number of microbial colonies growing on the substrate may be visualized, and, optionally, counted through cover sheet 20, if transparent.

As noted above, supporting substrate 12 can include one or more additional layers in addition to an oxygen scavenging layer. For example, device 10 can include substrate 30 as a component of supporting substrate 12. Substrate 30 can be a self-supporting, waterproof substrate, typically a relatively stiff material made of a waterproof or water impermeable material (i.e., does not absorb water) such as polyester, HDPE, COC, polypropylene, or polystyrene. The substrate may be oriented to further increase the stiffness. Other suitable waterproof materials include substrates such as paper containing a waterproof coating such as polyethylene.

Similarly, cover sheet 20 can include one or more layers in addition to, or as a substitute for, a single or multilayer film component including an oxygen scavenging material. For example, FIG. 1 illustrates an exemplary embodiment of the device of the invention in which cover sheet 20 includes a transparent film or sheet material to facilitate visualizing of microbial colonies present on the substrate. In addition, cover sheet 20 can be impermeable to bacteria and water vapor to avoid the risk of contamination and deterioration of the components of the culture device. One exemplary material for use as a cover sheet 20 is biaxially oriented polypropylene. As noted above, the cover sheet can be coated with a gelling agent and/or nutrients and/or an optional adhesive. Cover sheet 20 may further include a reinforcement layer, such as a nonwoven material, foam (e.g., a polystyrene foam), or film (e.g., a polycarbonate film), for additional support.

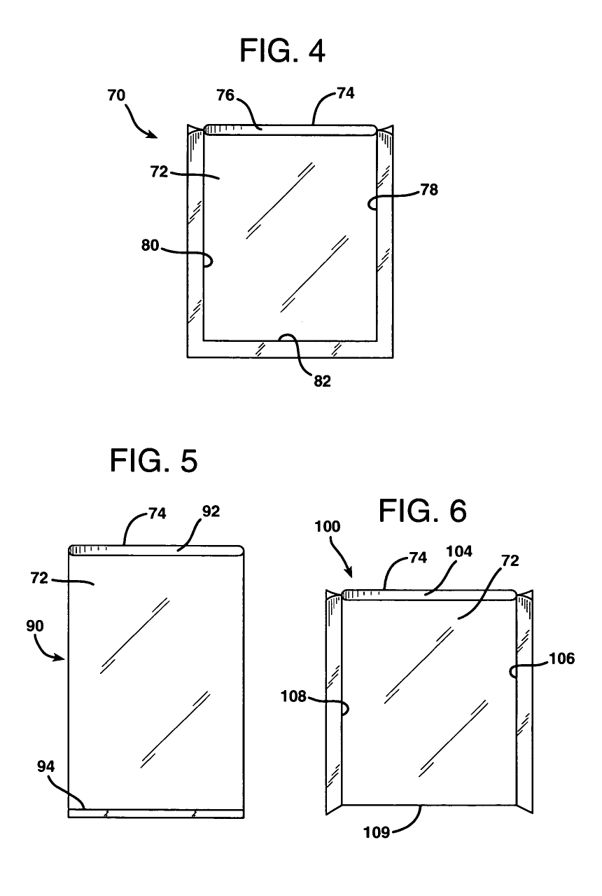

FIGS. 4, 5 and 6 illustrate various alternative embodiments of a culture device of the invention, in which the device is in the form of a container, such a pouch, bag, casing, or sheet formed from joined film pieces, at least one of which includes an oxygen scavenging material. In this aspect of the invention, the oxygen scavenging material can be in the form of a single film layer, monolayer film, or coating, any of which may be continuous or discontinuous. Alternatively the oxygen scavenging material can be in the form of a multilayer film, such as a three layer film 18 described above with reference to FIG. 2. The film component including an oxygen scavenging material can be heat sealed to itself or to another film, which can be the same or different from the oxygen scavenging film component. The film can be used as a component in a rigid, semi-rigid, or flexible product, and can also be adhered to another polymeric substrate and/or to a non-polymeric or non-thermoplastic substrate such as paper or metal.

FIG. 4 is a top view of one embodiment of a container in accordance with this aspect of the invention. FIG. 4 illustrates a pouch, designated generally as 70, made from two rectangular pieces of flexible film, 72 and 74, of the same dimensions, which are sealed to one another along three edges, leaving the unsealed fourth edges to form the open top, into which a product can be inserted. In FIG. 4, pouch 70 is illustrated in a substantially lay-flat position. Each of films 72 and 74 includes at least one film layer including an oxygen scavenging material, as described above. For example, each of films 72 and 74 can include a film or film layer comprising an oxygen scavenging material, bonded or sealed to at least one other film or film layer. Pouch 70 includes a pouch mouth 76, side seals 78 and 80 and end seal 82. Film 72 and 74 can also include other materials known in the art for the production of films for packaging applications. Either or both films 72 and 74 can include oxygen scavenging material.

Film layer 72 and 74 can each comprise a single film layer or coating, or monolayer film, or can include two or more film layers to provide a multilayer film, such as multilayer film 18 described above. Advantageously either or both film 72 and 74 is a multilayer film, typically including at least three layers, although the multilayer film can include fewer or more layers. The multilayer film typically includes at least an oxygen scavenging layer, an oxygen barrier layer, and an optional adhesive layer therebetween, such as layers 40, 42 and 44 described above with reference to FIG. 2.

In use, the oxygen scavenger of pouch 70 is activated by actinic radiation and a suitable culture device for growing anaerobic microorganisms is inoculated with a sample and the device inserted into pouch 70 via open end 76. For example, a culture device such as described above with respect to FIGS. 1-3 can be exposed to radiation to trigger the oxygen scavenging material, inoculated with a sample, and inserted into pouch 70 via opening 76. This aspect of the invention, however, is not limited to the use of culture devices such as described above with regard to FIGS. 1-3. For example, a thin film culture device as known in the art which does not include an oxygen scavenging material can be used, e.g., inoculated with a sample and placed within activated pouch 70. See U.S. Pat. Nos. 5,232,838; 4,565,783; 5,869,321; 5,443,963; 5,462,860; 5,958,675; 5,089,413; and 5,601,998, and U.S. patent application Publication 2002/0110906 A1, all incorporated herein by reference in their entirety, for examples of devices that do not inherently exhibit oxygen scavenging properties for use in this aspect of the invention. Other devices known in the art, such as Petri dishes and the like, can also be used in this aspect of the invention, so long as the inoculated culturing device is placed within a pouch including an oxygen scavenging material.

After the inoculated device is placed within pouch 70, open end 76 can be releasably sealed. Any suitable adhesive known in the art for releasably adhering film layers to one another can be placed along an inner portion of film layer 72, film layer 74, or both. Alternatively, pouch 70 may be heat sealed.

Alternatively, in this aspect of the invention, the device can be an end seal bag 90 as illustrated in a lay-flat position in FIG. 5. End seal bag 90 is made from film 72, in the form of a tube, with end seal bag 90 having an open top 92 and an end-seal 94. The respective sides of bag 90 are folds formed by the original tube. In yet another alternative embodiment of the invention, the device can be a side-seal bag, such as bag 100 illustrated in FIG. 6, also illustrated in a substantially lay-flat position. Side-seal bag 100 also includes a film layer 72 including an oxygen scavenging material. Side seal bag 100 includes an open top 104 and side seals 106 and 108. The bottom 109 of bag 100 is the fold formed by the original tube. Similar to the process described above with respect to the pouch of FIG. 4, an inoculated culture device is placed within bag 90 or bag 100, and open end 92 or 104, respectively, can be releasably sealed. Again, any suitable adhesive known in the art for releasably adhering film layers to one another can be placed along an inner portion of film layer(s) 72. Alternatively, the pouch may be heat sealed.

The culture devices of the invention can be triggered to scavenge oxygen by exposing the device to radiation. Generally the culture devices of the invention are triggered prior to inoculation of the device (for example, prior to inoculating a device such as that illustrated in FIG. 1) and/or prior to placement of an inoculated culture device within a container of the invention (such as that illustrated in FIGS. 4-6). Advantageously the culture device of the invention is triggered by exposing an exterior portion of the device to radiation. In this regard, the intervening layers(s) of the device that include the oxygen scavenging material is typically transparent to the triggering radiation. Intervening layers that are suitably transparent to actinic radiation do not include aromatic groups or highly chlorinated polymers, for example polyethylene terephthalate (PET), polyethylene naphthalate, saran (polyvinylidene dichloride or PVDC), saran coated PET, polystyrene, styrene copolymers, aromatic polyamides, and polycarbonate. One skilled in the art can readily determine which materials are suitably UV transparent; for example, most polyolefins, EVOH and aliphatic polyamides are sufficiently UV transparent to allow triggering of an oxygen scavenging layer through them. One advantage of the devices of the invention is that the devices, especially those including a high oxygen barrier structure, can be exposed to actinic radiation to initiate oxygen scavenging of oxygen in the interior of the device made in part or entirely from the oxygen scavenging material, while initiating oxygen scavenging that provides an active barrier to further ingress of oxygen from the exterior of the device.

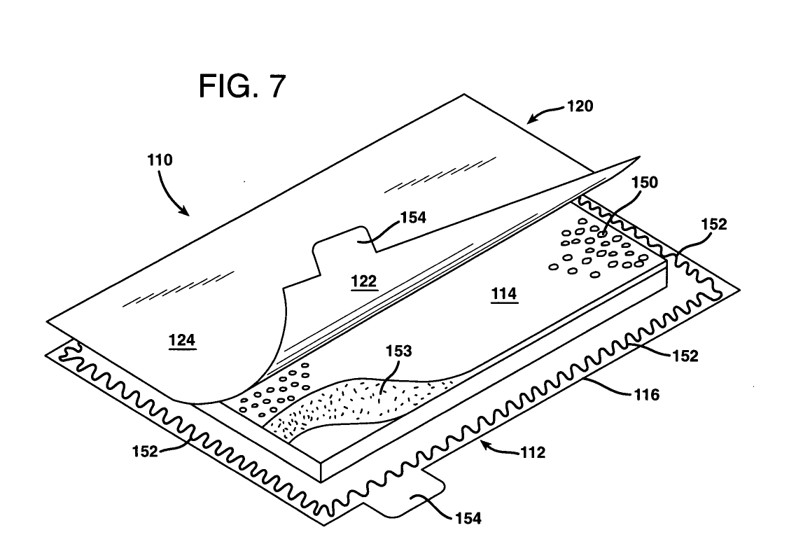

FIG. 7 discloses an alternative embodiment in which the culture device 110 is like the embodiment of FIG. 1 in all relevant respects, but further includes one or more adhesive or sealing regions along at least one edge of the device so that the cover sheet can releasably adhere to the supporting substrate. Thus, in FIG. 7 the anaerobic culture device 110 includes a supporting substrate 112 with an inner surface 114 opposing an outer surface 116. Culture device 110 also includes a cover sheet 120, which includes opposing inner surface 122 and outer surface 124. Cover sheet 120 is shown with an adhesive 152, such as adhesive 52 disclosed herein; such as a pressure sensitive adhesive, UV curable adhesive, adhesive of the type used in POST-IT™ notes available from 3M, or other suitable adhesive applied to at least one of, some of, or all four edges of the inner surface of supporting substrate 112. The adhesive can be a moisture activated adhesive. Alternatively, one or more edges of the supporting substrate 112 can include a region of a sealable material (either as a discrete strip or as a material forming an entire layer of the supporting substrate 112) which can form a peelable seal with corresponding edge portions of the inner surface 122 of cover sheet 120, through the application of e.g. heat, ultrasonic, or radio frequency (RF) sealing technology known to those of skill in the sealing art. The materials of the relevant edges of the supporting substrate and cover can be dissimilar chemically, but capable of forming a peelable (peel force less than 2 pounds/inch) seal when heat and pressure are applied. Optionally, the adhered or sealed region is reclosable.

More generally, at least one edge of the inner surface of supporting substrate 112 includes an adhesive or sealing region for releasably adhering cover sheet 120 to supporting substrate 112, although other(s) of the remaining edges can also include such adhesive or sealing regions. Alternatively, the adhesive or sealing region can be present on the inner surface 122 of cover 120, or on the inner surfaces of both the supporting substrate and cover sheet with respect to any selected edges of the device.

The adhesive or sealing region can be present in a continuous (as shown) or discontinuous pattern on the device.

Optional header tabs 154 can be formed, either as an integral part of cover sheet 120 and supporting substrate 112 respectively, or as discrete components thereof. These header tabs can be used to initiate peeling of the cover sheet away from the supporting substrate, and thus facilitate opening of the culture device.

One advantage of a peelable device is that it provides the capability of readily opening the device, e.g. to inoculate the culture medium, and then close the cover on top of the inoculated medium within the device. In advance of inoculation, and afterwards, the device can be stored in a closed condition.

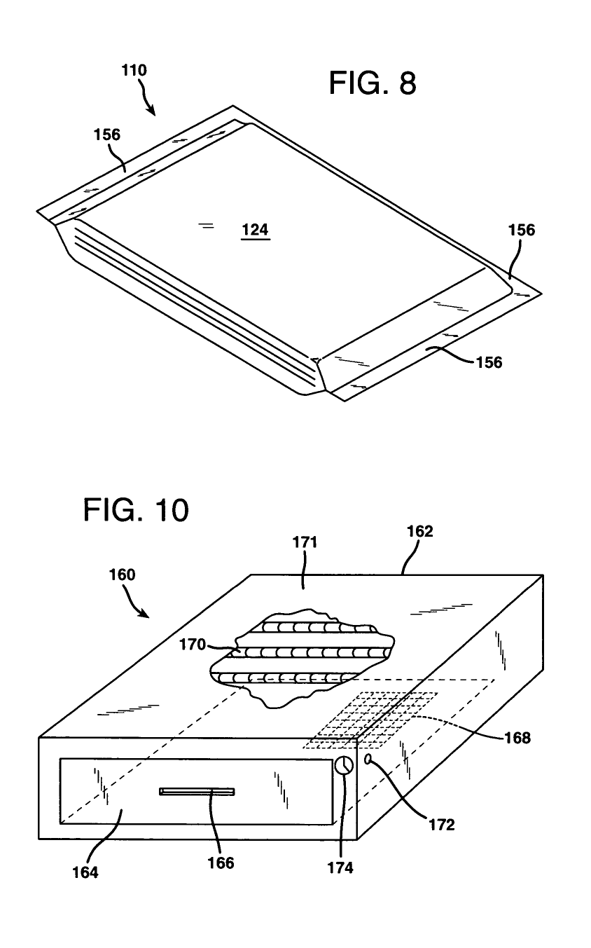

FIG. 8 discloses a top perspective view of the alternative embodiment of FIG. 7, in which the culture device is closed along closed edges 156. One side of the device is cut away for purposes of generically illustrating typical construction of the device, such as described with respect to FIG. 1.

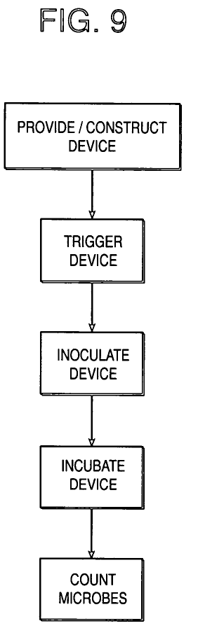

FIG. 9 shows a process flow diagram involving several steps. A culture device in accordance with the invention is provided (e.g. by purchase from a manufacturer) or constructed. The device is then triggered, as described herein, by e.g. UV-C light to activate the oxygen scavenger in the device. A culture medium in the device is then inoculated with a sample; the device is then beneficially closed and incubated. After an appropriate time, to be determined by a variety of conditions such as the nature of the inoculant, the construction of the device, etc., the microbes can be counted.

FIG. 10 shows a bench top triggering apparatus 160 useful in connection with the culture device of the invention. Triggering apparatus 160 includes an outer housing 162 that receives a drawer 164 having a handle 166. The drawer can carry one or more culture devices 168 of the invention. Device 168, or a plurality of devices 168, are placed on the planar floor of the drawer, and the drawer, with the device 168 placed thereon, is inserted into the housing 162. The housing 162 has suitably placed therein UV bulbs 170, or other source of actinic radiation. In FIG. 10, a partial cut away view of the top 171 of the housing 162 illustrates a bank of UV bulbs. The bulb array or other source of actinic radiation is activated to trigger the oxygen scavenger present in the device(s) 168. Appropriate power controls 172 and timer 174 can be utilized to control the dose of radiation. Suitable power connections, mounting brackets, etc. for use in connection with apparatus 160 are not shown, and will be evident to those skilled in the art upon review of this specification. After exposing each device 168 to the radiation, the drawer is opened, and the device or devices removed, and opened to inoculate the culture medium as described herein.

The culture devices of the invention can further include one or more indicators, e.g., colorants, incorporated into the structure thereof The indicator can be a photochromic material having a first appearance prior to exposure to radiation and a second appearance after exposure to radiation. The indicator thus can exhibit a first color prior to initiation of the oxygen scavenging material and at least a second color that is different from the first color following initiation to indicate to the user that the oxygen scavenging material is effectively triggered. Various photochromic materials that exhibit a color change upon exposure to radiation are known in the art and include without limitation leuco triphenylmethane cyanides, tetrazolium dyes, rhodamine dyes, photorome dyes, spiro pyran dyes, azo dyes, diazonium dyes, fluoran dyes, oxazolidine dyes and the like. These materials will typically be used at 0.1-2.0% with respect to the carrier material. The exact amount required to produce a distinct color change can readily be determined by experimentation. The colorant can be blended with one or more polymeric materials, and/or the oxygen scavenging composition prior to formation of the same into a film or other component for use in the construction of the culture devices of the invention. Alternatively, the colorant can be provided as a coating along one or more surfaces of the devices. In yet another alternative, the colorant can be provided in the form of printed indicia or a pattern along one or more surfaces of the devices. For example, the photochromic colorant can be incorporated in the grid pattern of FIG. 3. Alternatively, the colorant can be incorporated into the culture medium provided that it does not interfere with microbial growth. In general, the photochromic material can be incorporated anywhere in the device so long as the photochromic material does not interfere with some other function of the device and yet receives the actinic radiation that triggers the oxygen scavenger.

Other means are available to indicate that the oxygen scavenger has been suitably activated including materials that indicate the concentration of oxygen within the device directly. The oxygen indicator can be a luminescent compound that indicates the absence of oxygen inside of the device. Suitable oxygen indicators are disclosed in U.S. Pat. No. 6,689,438 (Kennedy et al.), incorporated herein as if set forth in full. Luminescent compounds appropriate as indicators for the present invention will display luminescence that is quenched by oxygen. More precisely, the indicators will luminesce upon exposure to their excitation frequency with an emission that is inversely proportional to the oxygen concentration. The indicator may be coated, laminated, or extruded onto another layer, or portion of another layer, within the device. Such a layer may be adjacent to the scavenging layer or separated from the scavenging layer by one or more other oxygen permeable layers. Suitable compounds include metallo derivatives of octaethylporphyrin, tetraphenylporphyrin, tetrabenzoporphyrin, the chlorins, or bacteriochlorins. Other suitable compounds include palladium coproporphyrin (PdCPP), platinum and palladium octaethylporphyrin (PtOEP, PdOEP), platinum and palladium tetraphenylporphyrin (PtTPP, PdTPP), camphorquinone (CQ), and xanthene type dyes such as erythrosin B (EB). Other suitable compounds include ruthenium, osmium and iridium complexes with ligands such as 2,2′-bipyridine, 1,10-phenanthroline, 4,7-diphenyl-1,10-phenanthroline and the like. Suitable examples of these include, tris(4,7,-diphenyl-1,10-phenanthroline)ruthenium(II) perchlorate, tris(2,2′-bipyridine)ruthenium(II) perchlorate, tris(1,10-phenanthroline)ruthenium(II) perchlorate, and the like.

It may be desirable to incorporate both a UV and an oxygen indicator into the culture device. The culture device can further include indicia printed on the inner surface of the supporting substrate or the inner surface of the cover sheet, for example, in the form of a grid pattern, to facilitate counting microorganism colony growth.

Such indicators would be located on the inner side of the oxygen barrier layers so as to indicate the oxygen level inside of the device.

### EXAMPLE 1

A 356 mm×285 mm pouch in accordance with the invention was made with an activated oxygen scavenging film (OS pouch). This pouch and a standard barrier pouch (P640B™, oxygen transmission rate=6.5 cm³ O₂/m²·day·atmosphere, available from Cryovac Inc.) were flushed with approximately 3100 cc of approximately 1% oxygen in nitrogen. An ANAEROPAK™ oxygen scavenging sachet made by Mitsubishi Gas Chemical Co. was placed in the standard barrier pouch and headspace oxygen levels were measured over time. Pouches were stored at ambient temperatures and headspace oxygen concentration was analyzed on day 0, 1, 4 and 6 using a Mocon PACCHECK™ 400. The results are summarized in Table 1 below.

TABLE 1

| | Day 0 | Day 0 | Day 1 | Day 1 | Day 4 | Day 6 |
|---|---|---|---|---|---|---|
| Time: | 14:30 | 16:30 | 8:30 | 10:00 | 11:00 | 13:30 |
| OS Pouch | 0.980 | 0.869 | 0.297 | 0.126 | 0.0008 | 0.0021 |
| (invention) | | | | | | |
| Barrier | 0.812 | 0.0427 | 0.0280 | 0.0242 | 0.0070 | 0.0011 |
| Pouch w/sachet | | | | | | |

The data in Table 1 shows that the OS pouch of the invention can produce a low oxygen environment comparable to an oxygen scavenging sachet.

### EXAMPLE 2

To determine the suitability of oxygen scavenging films to rapidly create an oxygen free environment, 20×20 cm pouches were made from three different oxygen scavenging films, designated as OS10000™, LDX 7594™, and LDX 8071. OS1000 and LDX 7594 are commercially available from Cryovac Inc.

These films are generically described below:

| | | | | | | | | |
|---|---|---|---|---|---|---|---|---|
| LDX | Olefin | Oxygen | Adhesive | Oxygen | Adhesive | Bulk | Adhesive | Oriented |
| 7594 | sealing | scavenging | layer | barrier | layer | layer | layer | substrate |
| | layer | layer | | layer | | | | layer |
| LDX | Olefin | Oxygen | Adhesive | Oxygen | Adhesive | Abuse | | |
| 8071 | sealing | scavenging | layer | barrier | layer | layer | | |
| | layer | layer | | layer | | | | |

Just prior to making the pouches the films were activated with a dose of ultraviolet C light (UV-C) about 700 mJ/cm². The pouches were heat sealed and inflated with 150 cc of room air and then placed in a 39° C. oven. The oxygen concentration in the headspace was determined as above. The results are shown in Table 2 below.

TABLE 2

| OS1000 | | LDX 7812 | | LDX 8071 | |
|---|---|---|---|---|---|
| Time (hrs) | % Oxygen | Time (hrs) | % Oxygen | Time (hrs) | % Oxygen |
| 0 | 20.600 | 0 | 20.6 | 0 | 20.600 |
| 7 | 0.002 | 7 | 0.99 | 7 | 0.717 |
| 22 | 0.000 | 22 | 0.000 | 22 | 0.000 |

The data in Table 2 shows that the oxygen scavenging films are capable of rapidly producing an anaerobic environment for culturing microbes.

### EXAMPLE 3

Anaerobic and lactic acid bacteria counts resulting from the use of conventional pour plate and Petri-film® methods are compared with those resulting from the use of a device accordance with the present invention that includes an oxygen scavenging film as a component. The following procedure was performed.

A sample of cooked turkey meat known to have high lactic acid bacterial counts was diluted 1:10 with peptone buffered water and agitated in a stomacher for one minute.

Serial dilutions of the turkey meat were made and plated on APT agar plates for lactic acid bacteria counts and standard methods agar for anaerobic counts. The plates are double layered with agar to create an anaerobic environment (pour plate method).

Serial dilutions of the turkey meat were made in MRS broth and plated on PETRI-FILM® aerobic plates for lactic acid bacteria counts. The plates were placed in a bag with oxygen barrier properties and an oxygen-absorbing sachet to create an anaerobic environment.

Serial dilutions of the turkey meat were made in peptone buffered water and plated on Petri-film® aerobic plates for anaerobic counts. These plates were also placed in a bag with oxygen barrier properties and an oxygen-absorbing sachet.

All of the above were incubated at 35° C. for 48 hours.

Two examples of devices in accordance with the present invention were prepared as follows. Two types of activated oxygen scavenging film (OS1000™ and OS2000™, commercially available from Cryovac, Inc.) were sandwiched around the layers of a PETRI-FILM® device so that bacteria present would be exposed to nutrients provided on the film, and environmental oxygen was scavenged over time. The inoculants are then plated onto these films. The oxygen scavenging film test samples were incubated 48 hours.

All plates were pulled from incubators after 48 hours and bacterial colonies are counted. The results in log CFU/g (“CFU” herein means coliform forming units) are set forth in Table 3 below.

TABLE 3

| | Lactic Acid Bacteria | Anaerobic |
|---|---|---|
| | Average | Bacteria Average |
| Pour Plate Method | 5.48 | 6.74 |
| PETRI-FILM ® Method with | 6.49 | 6.50 |
| sachet | | |
| Device with OS film component | 6.99 | 5.37 |
| OS2000 ™ (Invention) | | |
| Device with OS film component | 6.94 | 3.72 |
| OS1000 ™ (Invention) | | |

This example shows that oxygen scavenging film can produce a sufficiently anaerobic environment to culture anaerobic microorganisms. This can be accomplished without hermetically sealing the device.

### EXAMPLE 4

Anaerobic and lactic acid bacteria counts resulting from the use of PETRI-FILM® were compared with those resulting from the use of a method in accordance with the present invention that includes an oxygen scavenging film. The following procedure was performed.

Roast beef near the end of its shelf life was tested for anaerobic and lactic acid bacteria by inoculating PETRI-FILM with three dilutions in duplicate. The inoculated PETRI-FILM was placed either into a 320×205 mm P640B barrier pouch with an oxygen scavenging and CO₂ generating sachet (ANAEROPAK) from Mitsubishi Gas Chemical or into a 320×205 mm pouch made from activated LDX 7594. The following results were obtained:

TABLE 4

| | Anaerobic Bacteria | Lactic Acid Bacteria |
|---|---|---|
| Package Type | (log CFU/g) | (log CFU/g) |
| Activated LDX 7594 | 7.53 | 7.65 |
| P640B w/Sachet | 7.57 | 7.62 |

These results show that the method of the invention can be used to determine anaerobic microorganism counts without the use of a sachet.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. Although the invention herein is primarily concerned with devices for growing and counting anaerobic microorganisms, and finds particular benefit in that area, those skilled in the relevant art will appreciate that the invention in its various embodiments can be useful in the growth and counting of other microorganisms as well, such as aerobic microorganisms.

Various combinations of one or more additional layers or substrates can also be used in combination with the oxygen scavenging material. As a non-limiting example, FIG. 1 illustrates a substrate 30 as an optional additional component or layer of supporting substrate 12. Modifications and variations may be utilized without departing from the principles and scope of the invention, as those skilled in the art will readily understand.

Whether present as a monolayer film or a multilayer film, the film can have any total thickness desired, so long as the film provides the desired oxygen scavenging properties for the particular culturing operation in which the film is used. The films generally have a thickness ranging from about 0.5 to about 10 mil, although films with a thickness outside of this range can also be used in accordance with the present invention.

1. A device for growing microorganisms, comprising:
a) a supporting substrate having opposing inner and outer surfaces;
b) a cover sheet having opposing inner and outer surfaces affixed to at least one edge of the inner surface of said supporting substrate and positioned to cover at least a portion of said supporting substrate; and
c) media for supporting the growth of microorganisms;
wherein said device comprises an oxygen scavenger.

2. The device of claim 1, wherein at least one of said supporting substrate and said cover sheet comprises a monolayer film comprising said oxygen scavenger.

3. The device of claim 1, wherein at least one of said supporting substrate and said cover sheet comprises a multilayer film layer comprising:
a) a layer comprising said oxygen scavenger; and
b) an oxygen barrier layer.

4. The device of claim 3, wherein said multilayer film layer further comprises at least one adhesive layer positioned between and adhering said oxygen scavenging layer and said oxygen barrier layer.

5. The device of claim 1 wherein said media comprises at least one gelling agent and at least one nutrient.

6. The device of claim 5 wherein said gelling agent and said nutrient are adjacent the inner surface of at least one of said supporting substrate, and said cover sheet.

7. The device of claim 5 wherein said gelling agent and said nutrient comprise reconstitutable powder.

8. The device of claim 1 wherein said device further comprises an indicator to indicate exposure of the device to actinic radiation sufficient to trigger the oxygen scavenger.

9. The device of claim 8 wherein said indicator comprises a photochromic material having a first appearance prior to exposure to radiation and a second appearance after exposure to radiation.

10. The device of claim 8 wherein said indicator comprises a luminescent compound.

11. The device of claim 1 further comprising indicia printed on the inner surface of said supporting substrate or the inner surface of said cover sheet to facilitate counting microorganism colony growth.

12. The device of claim 11 wherein said indicia is in the form of a printed grid pattern.

13. The device of claim 1 wherein said supporting substrate comprises at least one film layer comprising said oxygen scavenger; and wherein said cover sheet comprises a transparent polymeric film.

14. The device of claim 1 wherein the microorganisms comprise anaerobic microorganisms.

15. An article for culturing microorganisms, comprising:
a) a container comprising at least one film layer comprising an oxygen scavenger; and
b) a device for growing microorganisms, the device comprising:
i) a supporting substrate having opposing inner and outer surfaces;
ii) a cover sheet having opposing inner and outer surfaces affixed to at least one edge of the inner surface of said supporting substrate and positioned to cover at least a portion of said supporting substrate; and
iii) culture media for supporting the growth of microorganisms;
wherein the device for growing microorganisms is enclosed by the container.

16. The article of claim 15 wherein said container comprises a multilayer film comprising
a) a layer comprising said oxygen scavenger; and
b) an oxygen barrier layer.

17. The article of claim 16 wherein said multilayer film layer further comprises at least one adhesive layer positioned between and adhering said oxygen scavenging layer and said oxygen barrier layer.

18. The article of claim 15 wherein said container is a pouch, an end-seal bag, or a side-seal bag.

19. The article of claim 15 wherein the culture media is present on the inner surface of said supporting substrate.

20. The article of claim 15 wherein at least one of said supporting substrate and said cover sheet comprises an oxygen scavenger.

21. The article of claim 15 wherein the microorganisms comprise anaerobic microorganisms.

22. A method for determining microorganism counts in a device comprising a culture media, wherein the device is enclosed in a discrete container comprising an oxygen scavenger, comprising the steps of:
a) exposing the container comprising the oxygen scavenger to actinic radiation at a dosage sufficient to trigger the oxygen scavenger;
b) inoculating the culture media of the device with a predetermined volume of aqueous test sample;
c) placing the device in said container;
d) closing the container;
e) incubating the device; and
f) counting the number of microorganism colonies growing on the culture media.

23. The method of claim 22 wherein the microorganisms comprise anaerobic microorganisms.

24. A method for determining microorganism counts, comprising the steps of:
a) exposing a device to actinic radiation at a dose sufficient to trigger an oxygen scavenger within the device, the device comprising
i) a supporting substrate having opposing inner and outer surfaces;
ii) a cover sheet having opposing inner and outer surfaces affixed to at least one edge of the inner surface of said supporting substrate and positioned to cover at least a portion of said supporting substrate; and
iii) culture media for supporting the growth of microorganisms;
b) inoculating the culture media of the device with a predetermined volume of aqueous test sample;
c) incubating the device; and
d) counting the number of microorganism colonies growing on the culture media.

25. The method of claim 24 wherein the microorganisms comprise anaerobic microorganisms.

26. A method of making an anaerobic culture media device useful for growing microorganisms comprising:
a) providing a supporting substrate having opposing inner and outer surfaces that incorporates a culture media on the inner surface;
b) providing a cover sheet having opposing inner and outer surfaces;
c) coating a layer of the cover sheet with adhesive and uniformly affixing a gelling agent and nutrient medium to the inner surface; and
d) affixing the cover sheet to at least one edge of the inner surface of said supporting substrate; the cover sheet positioned to cover at least a portion of said supporting substrate;
wherein at least one of the supporting substrate and cover sheet comprises an oxygen scavenger and an oxygen barrier.

27. The method of claim 26 wherein the microorganisms comprise anaerobic microorganisms.
